(19) United States
(12) Patent Application Publication
(10) Pub. No.: US 2010/0256047 A1
(43) Pub. Date: Oct. 7, 2010

(54) Analyte Measurement and Management Device and Associated Methods

(75) Inventors: Zara Sieh, Pleasanton, CA (US); David Horwitz, Los Altos, CA (US); David Price, Pleasanton, CA (US); Peter Krulevitch, Pleasanton, CA (US); Donna Savage, Rolling Hills Estates, CA (US); Robert Shartle, Arnold, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: LifeScan, Inc., Milpitas, CA (US)

(21) Appl. No.: 12/417,875

(22) Filed: Apr. 3, 2009

(51) Int. Cl. A61K 38/28; A61P 3/10; G06F 19/00

(52) U.S. Cl. 514/3; 702/19

(57) ABSTRACT

A method for measuring and managing an analyte (e.g., blood glucose) in a bodily fluid includes storing a therapeutic administration protocol in a memory module of an analyte measurement and management device and measuring the analyte in the bodily fluid sample using an analyte measurement module of the device. The method also includes calculating, with a processor module of the device, a recommended therapeutic agent dosage (for example, an insulin dosage) and a recommended administration time for user-activated delivery of the dosage by employing the therapeutic administration protocol. The method further includes displaying the recommended therapeutic agent dosage and administration time to a user on a visual display of the device, delivering a therapeutic agent dosage to the user via a user-activated therapeutic agent delivery device, and detecting the user-activated administration of the therapeutic agent using a delivery device communication module of the device. In addition, the method includes communicating the aforementioned detection to the processor module and/or memory module using the delivery device communication module. The method employs analyte measurement, memory, processor, and delivery device modules, as well as a visual display, and user interface that are integrated as a single hand-held unit.

### BACKGROUND OF THE INVENTION

Introduction and management of insulin therapy to a patient with Type 2 diabetes can be overwhelming to the patient and a burden to the provider due to the complexity of conventional methods and devices for doing so. Significant training of the patient may be necessary. The patient may need to learn, for example, various concepts and actions including hypoglycemia management, injections and the proper use insulin administration devices, as well as the mechanical, electronic, and software aspects of using a blood glucose meter. In addition, the patient must learn to follow the doctor's instructions in starting and adjusting insulin dosages on a regular basis (e.g. per meal, daily, 2× weekly, or weekly basis).

Detailed instructions as to the prescribed blood glucose testing and insulin titration protocol are typically written out by the health care professional or checked off on a piece of paper. Patients often keep handwritten logs in order to comply.

After getting onto insulin therapy, a the patient often times presents in a physician's office with poor glycemic control and the care provider (i.e., physician) can be left guessing as to whether the poor glycemic control is due to, for example, noncompliance, or whether increased intensification of insulin therapy is required, or a combination thereof.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements), of which:

FIG. 1 is a simplified plan view of an analyte measurement and management device according to an embodiment of the present invention;

FIG. 2 is a simplified block diagram illustrating the internal components of an analyte measurement and management device according to an embodiment of the present invention;

FIG. 3 is a flow chart illustrating a method of operating an analyte measurement device, during which a recommended therapeutic agent dosage and a recommended time for administration of the recommended therapeutic agent dosage are calculated, according to an embodiment of the present invention;

FIG. 4 is an exemplary flow chart illustrating a method of operating an analyte measurement device, in which a therapeutic agent type is entered and a list of administration protocols are displayed, according to an embodiment of the present invention;

FIG. 5 is an exemplary flow chart illustrating a method of operating an analyte measurement device, in which a user's health profile is entered and a recommended therapeutic agent and administration protocol are displayed, according to an embodiment of the present invention;

FIG. 6 is an exemplary flow chart illustrating a method of operating an analyte measurement device, in which an intensification administration protocol is selected, according to an embodiment of the present invention;

FIG. 7 is an exemplary flow chart illustrating a method of operating an analyte measurement device, in which a recommended administration protocol is reinitialized, according to an embodiment of the present invention;

FIG. 8 is a flow chart illustrating a method of operating an analyte measurement device, in which a user is reminded to test and administer therapeutic agent if confirmation of testing or administration is not received within a time window, according to an embodiment of the present invention;

FIG. 9 is a flow chart illustrating a method of operating an analyte measurement device, in which more than one therapeutic agent is selected and more than one recommended administration protocol is displayed, according to an embodiment of the present invention;

FIG. 10 is an exemplary flow chart illustrating a method of operating an analyte measurement device, in which reminders are displayed along with a compliance report, according to an embodiment of the present invention;

FIG. 11 illustrates a series of user interface screen images (displays) as can be used in methods according to various embodiments of the present invention;

FIG. 12 illustrates a user interface screen images, that assists a healthcare provider in selecting an administration protocol, that can be employed in methods according to an embodiment;

FIG. 13 illustrates a user interface image, in which a summary report is displayed, that can be employed in methods according to the present invention;

FIG. 14 illustrates an exemplary treat-to-target therapeutic administration protocol that can be used in embodiments of the present invention;

FIG. 15 illustrates another exemplary treat-to-target therapeutic administration protocol that can be employed in embodiments of the present invention;

FIG. 16 illustrates yet another exemplary treat-to-target protocol that can be utilized embodiments of the present invention;

FIG. 17 is a simplified block diagram of an analyte measurement and management device for use with a user-activated therapeutic agent delivery device according to an embodiment of the present invention; and

FIG. 18 is a flow diagram illustrating stages in a method according to an embodiment of the present invention.

### DETAILED DESCRIPTION OF EMBODIMENTS OF THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Embodiments of the present invention provide an analyte (e.g., blood glucose) measurement and management device and associated methods that simplify training and guide a patient regarding when to measure an analyte (i.e., to “test”) and how much and when to administer a therapeutic agent (such as insulin) in a simple and convenient manner and with a minimum of devices. Embodiments of the analyte measurement and management device and system are also beneficial to care providers (for example, physicians) by gathering, organizing and storing information that provides insight into how effective a patient is in following a prescribed analyte management regimen.

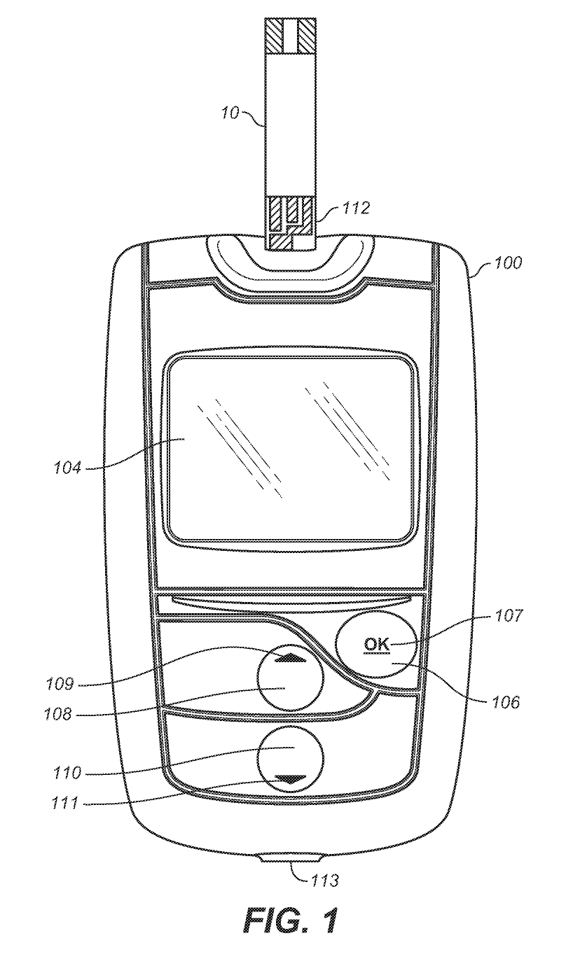

FIG. 1 illustrates an analyte measurement and management device 100 (also referred to herein for simplicity as a “meter,” an “analyte measurement device,” and a “testing device”), for testing (measuring or determining) and managing glucose levels in the blood of an individual. As is described further herein, analyte measurement and management device 100 is for use with a user-activated therapeutic agent delivery device. In this regard, the term “user-activated” refers to therapeutic delivery devices that require manual interaction between the device and a user (for example, by a user pushing a button on the device) to initiate a single therapeutic agent delivery event and that in the absence of such manual interaction deliver no therapeutic agent to the user. A non-limiting example of such a user-activated therapeutic agent delivery device is described in co-pending U.S. Provisional Application No. 61/040,024 (tentatively identified by Attorney Docket No. LFS-5180) and which is hereby incorporated in whole by reference.

Analyte measurement device 100 may include user interface buttons (106, 108, 110) for entry of data, navigation of menus, and execution of commands. Data can include values representative of analyte concentration, and/or information, which are related to the everyday lifestyle of an individual. Information, which is related to the everyday lifestyle, can include food intake, medication use, the occurrence of health check-ups and general health condition and exercise levels of an individual. Analyte measurement device 100 also may include display 104. Display 104 can be used to report measured glucose levels, and to facilitate entry of lifestyle related information.

Analyte measurement device 100 may include first user interface button 106, second user interface button 108, and third user interface button 110. User interface buttons 106, 108, and 110 facilitate entry and analysis of data stored in the testing device, enabling a user to navigate through the user interface displayed on display 104. User interface buttons 106, 108, and 110 include first marking 107, second marking 109, and third marking 111, which help in correlating user interface buttons to characters on display 104.

Analyte measurement device 100 can be turned on by inserting a test strip 10 into data port 112, by pressing and briefly holding first user interface button 106, or when data traffic is detected across data port 113. Analyte measurement device 100 can be switched off by removing the test strip 10, pressing and briefly holding first user interface button 106, navigating to and selecting a meter off option from a main menu screen, or by not pressing any buttons for a predetermined time. Display 104 can optionally include a backlight.

Data port 113 accepts a suitable connector attached to a connecting lead, thereby allowing analyte measurement device 100 to be linked to an external device such as a personal computer. Data port 113 can be any port that allows for transmission of data (serial or parallel) such as, for example, serial or parallel port in wired or wireless form. A personal computer, running appropriate software, allows entry and modification of set-up information (e.g. the current time, date, and language), and can perform analysis of data collected by analyte measurement device 100. In addition, the personal computer may be able to perform advanced analysis functions, and/or transmit data to other computers (i.e. over the internet) for improved diagnosis and treatment. Connecting analyte measurement device 100 with a local or remote computer facilitates improved treatment by health care providers.

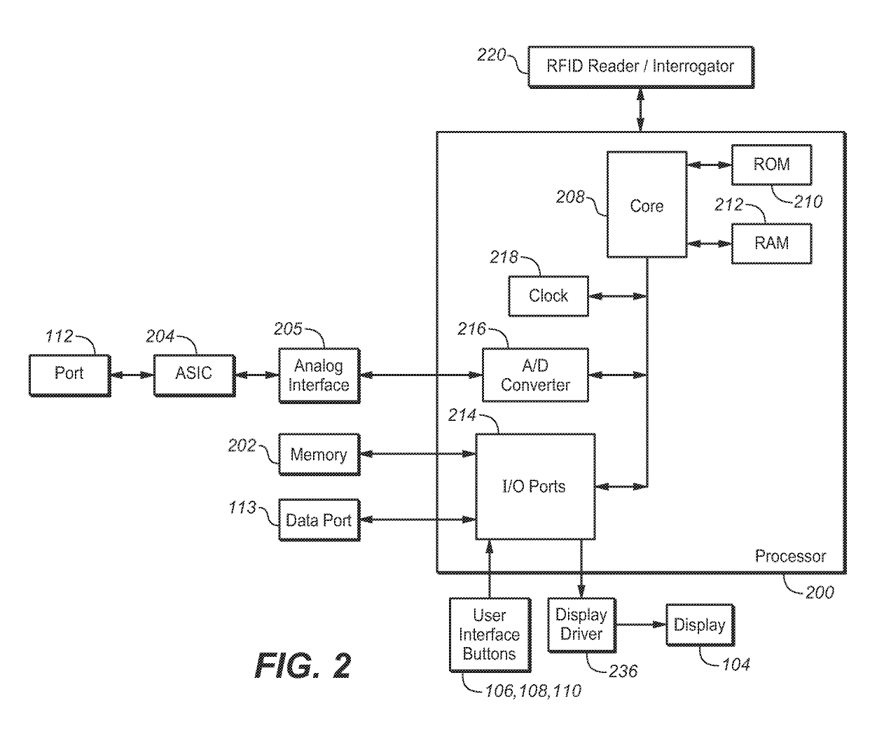

Referring to FIG. 2, an exemplary internal layout of analyte measurement device 100 is shown. Analyte measurement device 100 may include a processor 200, which in some embodiments described and illustrated herein is a 32-bit RISC microcontroller. The processor can be bi-directionally connected via I/O ports 214 to memory 202, which in some embodiments described and illustrated herein is an EEPROM. Also connected to processor 200 via I/O ports 214 are the data port 113, the user interface buttons 106, 108, and 110, and a display driver 236. Data port 113 can be connected to processor 200, thereby enabling transfer of data between memory 202 and an external device, such as a personal computer. User interface buttons 106, 108, and 110 are directly connected to processor 200. Processor 200 controls display 104 via display driver 236.

In embodiments described and illustrated herein, analyte measurement device 100 may include an Application Specific Integrated Circuit (ASIC) 204, providing electronic circuitry used in measurements of glucose level in blood that has been applied to a test strip 10 inserted into strip port 112. Analog voltages can pass to and from ASIC 204 by way of analog interface 205. Analog signals from analog interface 205 can be converted to digital signals by A/D converter 216. Processor 200 further comprises core 208, ROM 210 (containing computer code), RAM 212, and clock 218. In one embodiment, the processor 200 is configured (or programmed) to disable all of the user interface buttons except for a single button upon a display of an analyte value by the display unit such as, for example, during a time period after an analyte measurement. In an alternative embodiment, the processor 200 is configured (or programmed) to ignore any input from all of the user interface buttons except for a single button upon a display of an analyte value by the display unit.

In embodiments described and illustrated herein, analyte measurement device 100 may include a Radio Frequency Identification (RFID) Reader/Interrogator 220. In one embodiment, the reader/interrogator communicates with a passive RFID tag to identify the therapeutic agent delivery device. In an alternative embodiment the reader/interrogator communicates with a passive RFID tag within the therapeutic agent delivery device to detect administration of the therapeutic agent.

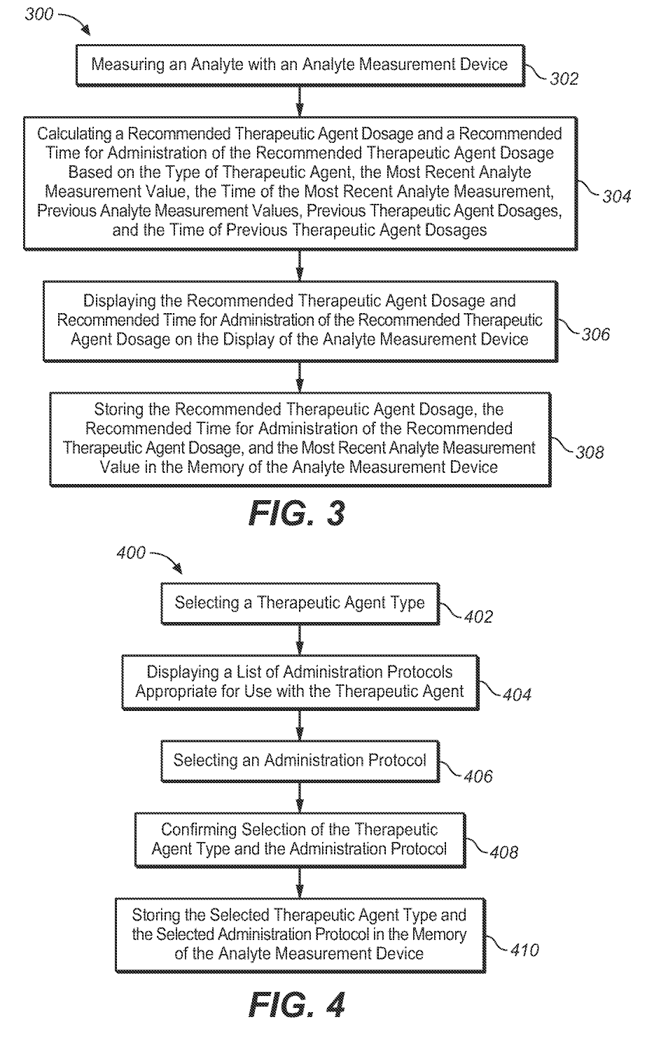

FIG. 3 is an exemplary flow chart illustrating a method of operating an analyte measurement device, according to an embodiment described and illustrated herein. Method 300 comprises steps 302, 304, 306, and 308. Step 302 includes measuring an analyte with the analyte measurement device. In an embodiment of the present invention, the analyte is measured using electrochemical techniques and the analyte is blood glucose. In other embodiments of the present invention the analyte is measured photo metrically, and the analyte is blood glucose. In other embodiments of the present invention the analyte is hemoglobin AIC, and the analyte is measured using immunoassay or electrochemical techniques. Step 304 includes calculating a recommended therapeutic agent dosage and a recommended time for administration of the recommended therapeutic agent dosage based on the type of therapeutic agent, the most recent analyte measurement value, the time of the most recent analyte measurement, previous analyte measurement values, previous therapeutic agent dosages, and the time of previous therapeutic agent dosages. In an embodiment of the present invention, the therapeutic agent is insulin and the analyte measurement is blood glucose. Step 306 includes displaying the recommended therapeutic agent dosage and recommended time for administration of the recommended therapeutic agent dosage on the display of the analyte measurement device. In an embodiment of the present invention, the display of the analyte measurement device is an LCD, and the recommended therapeutic agent dosage and recommended time for administration of the recommended therapeutic agent dosage are displayed on a user interface. Step 308 includes storing the recommended therapeutic agent dosage, the recommended time for administration of the recommended therapeutic agent dosage, and the most recent analyte measurement value in the memory of the analyte measurement device. In an embodiment of the present invention, the memory of the analyte measurement device includes a removable memory such as a single in-line memory module (SIMM) card. In an embodiment of the present invention, the method further comprises displaying a reminder to administer the recommended therapeutic agent dosage on the display of the analyte measurement device. In an embodiment of the present invention, reminders can be audible, such as a beep, or sensory, such as vibration. In an embodiment of the present invention, the method further comprises displaying a reminder to measure an analyte on the display of the analyte measurement device. In an embodiment of the present invention, the reminder reminds the user to measure their blood glucose. In an embodiment of the present invention, the reminder reminds the user to administer insulin. In an embodiment of the present invention, the method further comprises determining if the current time and date fall within a time window for analyte measurement; emitting an alarm if analyte measurement has not occurred within the time window; and storing a record of the alarm in the memory of the analyte measurement device. In an embodiment of the present invention, the current date and time is determined using the analyte measurement device internal clock. In an embodiment of the present invention, the method further comprises retrieving the recommended therapeutic agent dosage from the memory of the analyte measurement device; determining if the current time and date fall within a time window for administration of the recommended therapeutic agent dosage; emitting an alarm if administration of the recommended therapeutic agent dosage has not occurred within the time window for administration of the recommended therapeutic agent dosage; and storing a record of the alarm in the memory of the analyte measurement device. In an embodiment of the present invention, alarm records stored in the memory of the analyte measurement device can be used to establish compliance with recommended measurement and therapeutic agent administration protocols.

In an embodiment of the present invention, the therapeutic agent is long acting insulin and the time window is in the early morning or the late evening. In an embodiment of the present invention, the therapeutic agents include both long acting and rapid acting insulins and the time window for administering the long acting insulin is in the early morning or the late evening and the time window for administering the rapid acting insulin is premeal. In an embodiment of the present invention, the therapeutic agent is one of an oral antidiabetic agent, a GLP-1 agent, insulin and insulin mixes, or a combination thereof. In an embodiment of the present invention, the therapeutic agent is medication for metabolic management, hormonal therapies, oncology, pain management, regenerative medicine, or a combination thereof. In an embodiment of the present invention, the therapeutic agent is a medication used in the management of diabetes.

In an embodiment of the present invention, the analyte measurement device automatically displays the recommended therapeutic agent dosage after taking a blood glucose measurement, or after turning the analyte measurement device on. In an embodiment of the present invention, the recommended therapeutic agent dosage can be a function of at least one previous analyte measurement value if the measurement analyte value is greater than or less than preset thresholds. For example, if a blood glucose measurement is high the recommended insulin dosage may be increased, whereas if a blood glucose measurement is low the insulin dosage may be decreased. In an embodiment of the present invention, the analyte measuring device queries a user and upon user acceptance displays the recommended therapeutic agent dosage. The query can be in the form of a user interface prompt displayed on the analyte measurement device. User acceptance can include pressing a specific user interface button. In an embodiment of the present invention, the recommended therapeutic agent dosage is displayed in the form of units of insulin.

In an embodiment of the present invention, the recommended therapeutic agent dosage is displayed to a user in the format of user button pushes on the associated user-activated therapeutic agent delivery device. For example, such button pushes can be used to actuate the delivery of a predetermined amount of therapeutic agent by displacement from the user-activated therapeutic agent delivery device. A non-limiting example of such a user-activated therapeutic delivery device is described in the aforementioned U.S. Provisional Patent Application No. 61/040,024 (tentatively identified by Attorney Docket No. LFS-5180).

In an embodiment of the present invention, a user can toggle between displaying the recommended therapeutic agent dosage in the form of insulin units or button pushes. Toggling between insulin units and button pushes can be accomplished by way of the analyte measurement device user interface. In an embodiment of the present invention, the recommended therapeutic agent dosage is displayed in graphical form. Graphical forms can include column, bar, line, pie, circles, and lights. In an embodiment of the present invention, the recommended therapeutic agent dosage is presented to a user in audio form by an audio module of the testing device. In an embodiment of the present invention, the recommended therapeutic agent dosage does not exceed a preset maximum daily dosage. For example, a maximum daily dosage of insulin may be entered into the analyte measurement device, and subsequently limit the daily recommended therapeutic agent dosage. In an embodiment of the present invention, a time stamp for the analyte measurement is used to determine if the measurement is pre-breakfast, pre-lunch, pre-dinner, or pre-snack. For example, if the analyte measurement is performed at 7:00 am, it could be considered to be pre-breakfast, while a test performed at 5:00 pm could be considered to be pre-dinner. In an embodiment of the present invention, the method further comprises prompting a user to confirm that the measurement is pre-breakfast, pre-lunch, pre-dinner, or pre-snack. In an embodiment of the present invention, the method further comprises prompting a user to confirm that the measurement is pre-breakfast, pre-lunch, pre-dinner, or pre-snack; and, prompting the user to enter a start time of the most recent meal or snack if the meal was not pre-breakfast, pre-lunch, or pre-dinner. For example, if a measurement occurs outside the preset windows for breakfast, lunch, dinner, than the specific start time of a snack can be entered.

In an embodiment of the present invention, the method further comprises retrieving the recommended therapeutic agent dosage from the memory of the analyte measurement device; displaying the recommended therapeutic agent dosage and the recommended time for administration of the recommended therapeutic agent dosage on the display of the analyte measurement device; prompting a user to confirm administration of the recommended therapeutic agent if the current time and date is approximately equal to the recommended time for administration of the recommended therapeutic agent dosage; pressing at least one of the user interface buttons to confirm administration of therapeutic agent; and storing a record of the administration of therapeutic agent in the memory of the analyte measurement device. In an embodiment of the present invention, the analyte measurement device is a blood glucose meter, the therapeutic agent is insulin, the administration is performed with an insulin dosage device, and the dosage is confirmed by pressing a user interface button on the blood glucose meter. In an embodiment of the present invention, the method further comprises prompting a user to enter the amount of therapeutic agent administered if the amount of therapeutic agent administered differs from the recommended therapeutic agent dosage. For example, if the recommended dosage is 4 units, and only 3 units are injected, than the user would enter 3 units. In an embodiment of the present invention, the method further comprises prompting a user to enter the amount of therapeutic agent administered if the amount of therapeutic agent administered differs from the recommended therapeutic agent dosage; and, prompting the user to confirm the amount of therapeutic agent administered. Confirming the actual dosage increases the accuracy of dosage recommendations. In an embodiment of the present invention, the method further comprises prompting a user to enter the amount of therapeutic agent administered if the amount of therapeutic agent administered differs from the recommended therapeutic agent dosage; prompting the user to confirm the amount of therapeutic agent administered; and, storing the amount of therapeutic agent administered in the memory of the analyte measurement device. As mentioned previously, the memory of the analyte measurement device may include a removable portion, such as a SIMM card. In an embodiment of the present invention, the method further comprises activating a reporting summary; calculating the percentage of actual versus recommended analyte measurements and the percentage of actual versus recommended therapeutic agent dosages; and displaying the percentages. Reporting summaries are useful in accessing conformance to recommended protocols, and are particularly useful in communicating with health care practitioners. In an embodiment of the present invention, the method further comprises calculating and displaying an analyte measurement average for a weekly, monthly, quarterly, yearly, or 6 week time period. In an embodiment of the present invention, the method further comprises calculating a percentage of out-of-range high and out-of-range low analyte measurements over a period of time, and displaying the percentage of out-of-range high and out-of-range low analyte measurements and time period. High and low ranges can be preset on the measurement device or set by the user or a health care practitioner, and are useful in managing conditions such as diabetes. In an embodiment of the present invention, the method further comprises activating a reporting summary; calculating the percentage of actual versus recommended analyte measurements and the percentage of actual versus recommended therapeutic agent dosages over a period of time; and displaying the percentages and period of time. In an embodiment of the present invention, the method further comprises activating a reporting summary; calculating the percentage of actual versus recommended analyte measurements and the percentage of actual versus recommended therapeutic agent dosages; activating a downloading function; downloading data and reports from the analyte measurement device; confirming completion of the download; and storing the downloaded data and reports in the memory of an external device. External devices include personal computers, network computer systems, external removable memory readers, PDAs, and mobile phones. In an embodiment of the present invention, the method further comprises uploading the downloaded data into a database linked to insurance incentives, disease management, or motivational programs. In an embodiment of the present invention, the method further comprises uploading the downloaded data into a database linked to pay-for-performance programs. In an embodiment of the present invention, insurance incentives, motivational programs, and pay-for-performance programs can be accessed via the internet. In an embodiment of the present invention, the method further comprises uploading the downloaded data into a database linked to clinical data registries.

In an embodiment of the present invention, the method further comprises receiving at least one signal from a dosage device confirming administration of therapeutic agent; and storing a record of the administration of therapeutic agent in the memory of the analyte measurement device. Furthermore, methods according to the present invention can include steps of retrieving a recommended therapeutic agent dosage and associated recommended administration time from the memory (also referred to herein as a memory module), and displaying such a retrieved recommended therapeutic agent dosage and administration time to user on the visual display of the analyte measurement device. In an embodiment of the present invention, the signal is a wireless signal such as Bluetooth or radio-frequency identification (RFID). In an embodiment of the present invention, the dosage device is a pump or a pen. In an embodiment of the present invention, the RFID component in the dosage device is passive and the RFID component in the analyte measurement device is active. In an embodiment of the present invention, the RFID component in the dosage device is powered by receiving signals from the analyte measurement device. In an embodiment of the present invention, the dosage device includes a passive, active, or semi-passive radio-frequency tag. In an embodiment of the present invention, the method further comprises storing the amount of therapeutic agent remaining in the dosage device in the memory of the analyte measurement device. In an embodiment of the present invention, the method further comprises alerting a user if the amount of therapeutic agent remaining in the dosage device is less than the amount required for a preset number of dosages or expected daily dosage. In an embodiment of the present invention, the method further comprises displaying the amount of therapeutic agent remaining in the dosage device in the form of units, days, or graphs. In an embodiment of the present invention, the signal can include information related to therapeutic agent type, cartridge type, cartridge volume, and type of dosage device. For example, an insulin pump could send a signal to the analyte measurement device that includes information in respect to type of insulin being used, the type of pump cartridge, the volume of the pump cartridge, the type of pump, and the associated bolus increment per button push (for example 1 button push is equivalent to 3 units). In an embodiment of the present invention, the method further comprises of using the associated bolus increment per button push as input into the protocol algorithm. In an embodiment of the present invention, the method further comprises displaying the amount of therapeutic agent remaining in the dosage device after receiving the signal. In an embodiment of the present invention, the method further comprises of displaying the remaining number of button pushes necessary to complete the recommended dosage. In an embodiment of the present invention, the method further comprises sending a signal from the analyte measurement device to the dosage device to lock down the dosage device if the amount of therapeutic agent delivered exceeds a preset maximum for a preset time window. For example, if the daily maximum dosage is exceeded, a signal can be sent from the analyte measurement device to the pump to stop delivering insulin until the next day. In an embodiment of the present invention, the method further comprises sending a signal from the analyte measurement device to multiple dosage devices to stop delivering therapeutic agent if the amount of therapeutic agent delivered exceeds a preset maximum for a preset time window. In an embodiment of the present invention, the analyte measurement device can determine which form of therapeutic agent dosage units to display based upon the signal from the dosage device. In an embodiment of the present invention, the analyte measurement device can provide an alarm if a signal is received from a dosage device outside a preset time window. In an embodiment of the present invention, the method further comprises activating a reporting summary; calculating the percentage of actual versus recommended analyte measurements and the percentage of actual versus recommended therapeutic agent dosages; and displaying the percentages. In an embodiment of the present invention, the method further comprises calculating and displaying an analyte measurement average for a weekly, monthly, quarterly, yearly, or 6 week time period. In an embodiment of the present invention, the method further comprises calculating a percentage of out-of-range high and out-of-range low analyte measurements over a period of time; and, displaying the percentage of out-of-range high and out-of-range low analyte measurements and time period. In an embodiment of the present invention, the method further comprises activating a reporting summary; calculating the percentage of actual versus recommended analyte measurements and the percentage of actual versus recommended therapeutic agent dosages over a period of time; and displaying the percentages and period of time. In an embodiment of the present invention, the method further comprises activating a reporting summary; calculating the percentage of actual versus recommended analyte measurements and the percentage of actual versus recommended therapeutic agent dosages; activating a downloading function; downloading data and reports from the analyte measurement device; confirming completion of the download; and storing the downloaded data and reports in the memory of an external device. In an embodiment of the present invention, the method further comprises uploading the downloaded data into a database linked to insurance incentives, disease management, or motivational programs. In an embodiment of the present invention, the method further comprises uploading the downloaded data into a database linked to pay-for-performance programs. In an embodiment of the present invention, the method further comprises uploading the downloaded data into a database linked to clinical data registries.

FIG. 4 is an exemplary flow chart illustrating a method of operating an analyte measurement device, according to an embodiment described and illustrated herein. Method 400 comprises steps 402, 404, 406, 408, and 410. Step 402 includes selecting a therapeutic agent type. In an embodiment of the present invention, step 402 includes selecting a type of insulin. Step 404 includes displaying a list of administration protocols appropriate for use with the therapeutic agent. In an embodiment of the present invention, step 404 includes displaying a list of administration protocols that include measurement frequency, dosage frequency, and dosage amounts. Step 406 includes selecting an administration protocol. Step 408 includes confirming selection of the therapeutic agent type and the administration protocol. Step 410 includes storing the selected therapeutic agent type and the selected administration protocol in the memory of the analyte measurement device. In an embodiment of the present invention, the administration protocol is selected by way of a user interface menu. In an embodiment of the present invention, selecting the administration protocol includes entering a passcode, preventing inadvertent changes to the administration protocol. In an embodiment of the present invention, selecting a therapeutic agent type is initiated by inserting a hardware key into the analyte measurement device. In an embodiment of the present invention, a hardware key is inserted into the strip port connector or the data port to initiate selection of a therapeutic agent type. In an embodiment of the present invention, selecting a therapeutic agent type and administration protocol is initiated as a result of an analyte value such as an HbA1c value being in a preset range or a series of analyte measurement values, such as blood glucose values, being in a preset range.

In an embodiment of the present invention, the administration protocol may include one or more initiation, titration, and testing regimens. In an embodiment of the present invention, the method further comprises selecting a time zone on the analyte measurement device. In an embodiment of the present invention, the method further comprises confirming a recommended not-to-exceed daily dosage of therapeutic agent. In an embodiment of the present invention, the method further comprises entering a time zone and approximate time windows for meals, snacks, wake-up, and bedtime; and, storing the time zone and approximate time windows for meals, snacks, wake-up, and bedtime in the memory of the analyte measurement device. In an embodiment of the present invention, the method further comprises accepting or modifying the time zone and approximate time windows for meals, snacks, wake-up, and bedtime; and, storing the time zone and approximate time windows for meals, snacks, wake-up, and bedtime in the memory of the analyte measurement device. In an embodiment of the present invention, the method further comprises initiating an administration protocol updating function, downloading an updated administration protocol; confirming completion of the download, selecting the updated administration protocol, displaying a summary of the updated administration protocol, and storing the updated administration protocol in the memory of the analyte measurement device. Updates ensure the use of the most up-to-date protocols and regimens. In an embodiment of the present invention, the downloading can occur wirelessly, through a USB or other physical connection, or through connection to a removable memory card inserted into the analyte measurement device. In an embodiment of the present invention, the analyte measurement device can be linked electronically to a network computer and be identified by a software code unique to the analyte measurement device. In an embodiment of the present invention, initiating administration protocol updating occurs automatically or when activated by a user. For example, updating can occur automatically when connecting the analyte measurement device to a network, or can be manually activated by way of the user interface. In an embodiment of the present invention, a user confirms initiation of the administration protocol updating function.

In an embodiment of the present invention, the method further comprises activating a reporting summary; calculating the percentage of actual versus recommended analyte measurements and the percentage of actual versus recommended therapeutic agent dosages; and displaying the percentages. In an embodiment of the present invention, the method further comprises calculating and displaying an analyte measurement average for a weekly, monthly, quarterly, yearly, or 6 week time period. In an embodiment of the present invention, the method further comprises calculating a percentage of out-of-range high and out-of-range low analyte measurements over a period of time; and, displaying the percentage of out-of-range high and out-of-range low analyte measurements and time period.

In an embodiment of the present invention, the method further comprises activating a reporting summary; calculating the percentage of actual versus recommended analyte measurements and the percentage of actual versus recommended therapeutic agent dosages over a period of time; and displaying the percentages and period of time. In an embodiment of the present invention, the method further comprises activating a reporting summary; calculating the percentage of actual versus recommended analyte measurements and the percentage of actual versus recommended therapeutic agent dosages; activating a downloading function; downloading data and reports from the analyte measurement device; confirming completion of the download; and storing the downloaded data and reports in the memory of an external device. In an embodiment of the present invention, the method further comprises uploading the downloaded data into a database linked to insurance incentives, disease management or motivational programs. In an embodiment of the present invention, the method further comprises uploading the downloaded data into a database linked to pay-for-performance programs. In an embodiment of the present invention, the method further comprises uploading the downloaded data into a database linked to clinical data registries.

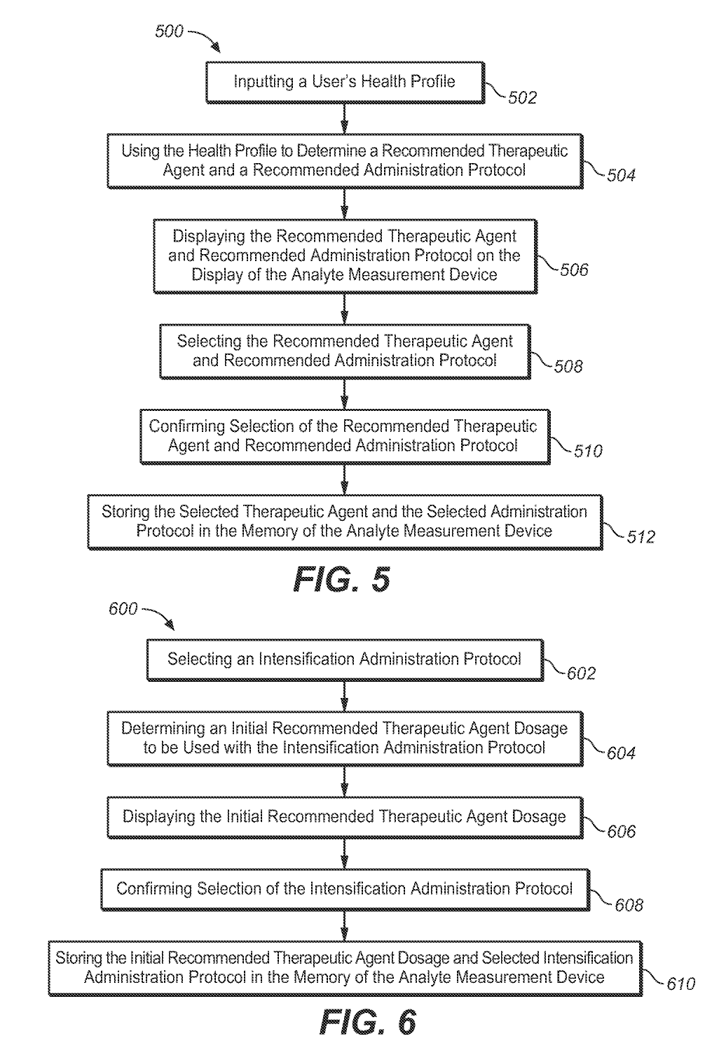

FIG. 5 is an exemplary flow chart illustrating a method of operating an analyte measurement device, according to an embodiment described and illustrated herein. Method 500 comprises steps 502, 504, 506, 508, 510, and 512. Step 502 includes inputting a user's health profile. Step 504 includes using the health profile to determine a recommended therapeutic agent and a recommended administration protocol. Step 506 includes displaying the recommended therapeutic agent and recommended administration protocol on the display of the analyte measurement device. Step 508 includes selecting the recommended therapeutic agent and recommended administration protocol. Step 510 includes confirming selection of the recommended therapeutic agent and recommended administration protocol. Step 512 includes storing the selected therapeutic agent and the selected administration protocol in the memory of the analyte measurement device. In an embodiment of the present invention, the user's health profile includes lifestyle and eating habits information. In an embodiment of the present invention, the user's health profile includes the largest meal size the patient consumes. In an embodiment of the present invention, the user's health profile can include previous blood glucose results, hemoglobin AIC results, weight, fasting glucose, or the user's tolerance to glucose. In an embodiment of the present invention, the method further comprises customizing the recommended administration protocol by setting an analyte measurement frequency or adjusting the therapeutic agent dosing. In an embodiment of the present invention, the method further comprises measuring an analyte with the analyte measurement device; calculating a starting therapeutic agent dosage based on the user's weight and the therapeutic agent initiation dosage multiplier; displaying the recommended starting therapeutic agent dosage and recommended time for therapeutic agent administration on the display of the analyte measurement device; and storing the recommended therapeutic agent dosage, the recommended time for therapeutic agent administration, and the current analyte measurement value into the memory of the analyte measurement device. If desired, methods and devices according to embodiments of the present invention can be configured to allow user confirmation, customization and/or acceptance of protocols and any recommendations thereof. In an embodiment of the present invention, the method further comprises initiating an administration protocol updating function; downloading an updated administration protocol, confirming completion of the download, selecting the updated administration protocol, displaying a summary of the updated administration protocol, and storing the updated administration protocol in the memory of the analyte measurement device. In an embodiment of the present invention, the downloading can occur wirelessly, through a USB or other physical connection, or through connection to a memory card inserted into the analyte measurement device. In an embodiment of the present invention, the analyte measurement device can be linked electronically to a network computer and be identified by a software code unique to the analyte measurement device. In an embodiment of the present invention, initiating administration protocol updating occurs automatically or when activated by a user. In an embodiment of the present invention, a user confirms initiation of the administration protocol updating function. In an embodiment of the present invention, the method further comprises activating a reporting summary, calculating a percentage of actual versus recommended analyte measurements and a percentage of actual versus recommended therapeutic agent dosages; and displaying the percentages.

In an embodiment of the present invention, the method further comprises calculating and displaying an analyte measurement average for a weekly, monthly, quarterly, yearly, or 6 week time period. In an embodiment of the present invention, the method further comprises calculating a percentage of out-of-range high and out-of-range low analyte measurements over a period of time; and, displaying the percentage of out-of-range high and out-of-range low analyte measurements and time period. In an embodiment of the present invention, the method further comprises activating a reporting summary, calculating the percentage of actual versus recommended analyte measurements and the percentage of actual versus recommended therapeutic agent dosages over a period of time; and displaying the percentages and period of time. In an embodiment of the present invention, the method further comprises activating a reporting summary, calculating a percentage of actual versus recommended analyte measurements and a percentage of actual versus recommended therapeutic agent dosages, activating a downloading function; downloading data and reports from the analyte measurement device, confirming completion of the download, and storing the downloaded data and reports in the memory of an external device. In an embodiment of the present invention, the method further comprises uploading the downloaded data into a database linked to insurance incentives, disease management or motivational programs. In an embodiment of the present invention, the method further comprises uploading the downloaded data into a database linked to pay-for-performance programs. In an embodiment of the present invention, the method further comprises uploading the downloaded data into a database linked to clinical data registries.

FIG. 6 is an exemplary flow chart illustrating a method of operating an analyte measurement device, according to an embodiment described and illustrated herein. Method 600 comprises steps 602, 604, 606, 608, and 610. Step 602 includes selecting an intensification administration protocol. Step 604 includes determining an initial recommended therapeutic agent dosage to be used with the intensification administration protocol. Step 606 includes displaying the initial recommended therapeutic agent dosage. Step 608 includes confirming selection of the intensification administration protocol. Step 610 includes storing the initial recommended therapeutic agent dosage and selected intensification administration protocol in the memory of the analyte measurement device. In an embodiment of the present invention, the intensification administration protocol is suggested after inputting the user's existing administration protocol. In an embodiment of the present invention, the intensification administration protocol is automatically suggested by the analyte measurement device if analyte measurements are high. In an embodiment of the present invention, the intensification administration protocol includes the use of short acting and long acting insulin.

In an embodiment of the present invention, the intensification administration protocol includes switching from long acting insulin to premixed insulin. In an embodiment of the present invention, the intensification administration protocol includes switching from premixed insulin to short acting insulin and long acting insulin. In an embodiment of the present invention, the intensification administration protocol includes switching from one therapeutic agent to another. In an embodiment of the present invention, the intensification administration protocol includes the use of one or more therapeutic agents. In an embodiment of the present invention, the method further comprises notifying the user that a new intensification administration protocol has been implemented; and, displaying times to conduct analyte measurements, times to administer therapeutic agent, and type of therapeutic agent to administer. In an embodiment of the present invention, the method further comprises querying the user as to whether reminders or alarms should be displayed if analyte testing or therapeutic agent administration does not occur as specified in the intensification administration protocol. In an embodiment of the present invention, the method further comprises displaying post-meal analyte measurement reminders at 1, 2, 3, and 4 hours after meals. In an embodiment of the present invention, reminders or alarms can be automatically or manually disabled. In an embodiment of the present invention, the method further comprises displaying a report summarizing the data related to the intensification administration protocol and at least one previous administration protocol.

In an embodiment of the present invention, the method further comprises initiating an intensification administration protocol updating function, downloading an updated intensification administration protocol, confirming completion of the download, selecting the updated intensification administration protocol, displaying a summary of the updated intensification administration protocol, and storing the updated intensification administration protocol in the memory of the analyte measurement device. In an embodiment of the present invention, the downloading can occur wirelessly, through a USB or other physical connection, or through connection to a memory card inserted into the analyte measurement device. In an embodiment of the present invention, the analyte measurement device can be linked electronically to a network computer and be identified by a software code unique to the analyte measurement device. In an embodiment of the present invention, initiating administration protocol updating occurs automatically or when activated by a user. In an embodiment of the present invention, a user confirms initiation of the administration protocol updating function. In an embodiment of the present invention, the method further comprises activating a reporting summary function of the device, calculating a percentage of actual versus recommended analyte measurements and a percentage of actual versus recommended therapeutic agent dosages, and calculating average premeal and 2 hr postmeal analyte values by mealtime (like breakfast, lunch and dinner).

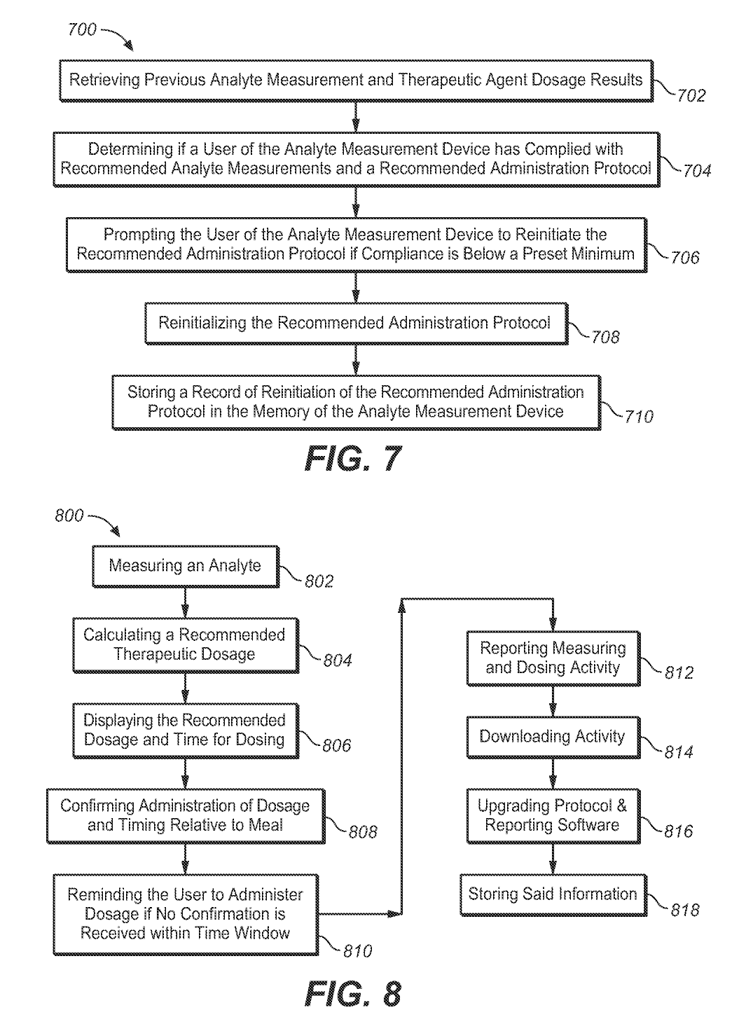

FIG. 7 is an exemplary flow chart illustrating a method of operating an analyte measurement device, according to an embodiment described and illustrated herein. Method 700 comprises steps 702, 704, 706, 708, and 710. Step 702 includes retrieving previous analyte measurement and therapeutic agent dosage results. In an embodiment of the present invention, previous analyte measurement and therapeutic agent dosage results are retrieved from the analyte measurement device's memory, or from a removable memory that is coupled with the analyte measurement device. Step 704 includes determining if a user of the analyte measurement device has complied with recommended analyte measurements and a recommended administration protocol. Compliance may include making analyte measurements and therapeutic agent dosages within specified time windows. Step 706 includes prompting the user of the analyte measurement device to reinitiate the recommended administration protocol if compliance is below a preset minimum.

Step 708 includes reinitializing the recommended administration protocol. Step 710 includes storing a record of reinitiation of the recommended administration protocol in the memory of the analyte measurement device. In an embodiment of the present invention, the method further comprises prompting the user to enter a reason for noncompliance. In an embodiment of the present invention, the method further comprises suggesting to the user that they contact a healthcare provider prior to reinitializing the recommended administration protocol if the reason for noncompliance is illness. In an embodiment of the present invention, a healthcare provider can preset compliance limits. In an embodiment of the present invention, the analyte measurement device can automatically reinitialize the recommended administration protocol if the user is noncompliant in respect to analyte measurements or therapeutic agent dosages. In an embodiment of the present invention, the analyte measurement device can automatically reinitialize the recommended administration protocol if the user was noncompliant for a preset time period.

In an embodiment of the present invention, the analyte measurement device can automatically continue the recommended administration protocol if the user was noncompliant for less than a preset time period. In an embodiment of the present invention, the analyte measurement device can automatically disable the recommended administration protocol upon noncompliance. In an embodiment of the present invention, the recommended administration protocol can be restarted. In an embodiment of the present invention, the method further comprises sending an alert to a health care practitioner that non-compliance has occurred.

FIG. 8 is an exemplary flow chart illustrating a method of operating an analyte measurement device, according to an embodiment described and illustrated herein. Method 800 comprises steps 802, 804, 806, 808, 810, 812, 814, 816, and 818. Step 802 includes measuring an analyte with an analyte measurement device. Step 804 includes calculating a recommended therapeutic dosage. Step 806 includes displaying the recommended dosage and time for dosing. Step 808 includes confirming administration of dosage and timing relative to a meal. Step 810 includes reminding the user to administer dosage if no confirmation is received within a time window. Step 812 includes reporting measuring and dosing activity. Step 814 includes downloading measurement and dosing activity. Step 816 includes upgrading the protocol & reporting software. Step 818 includes storing measurement, dosage, and reporting information in the memory of the analyte measurement device.

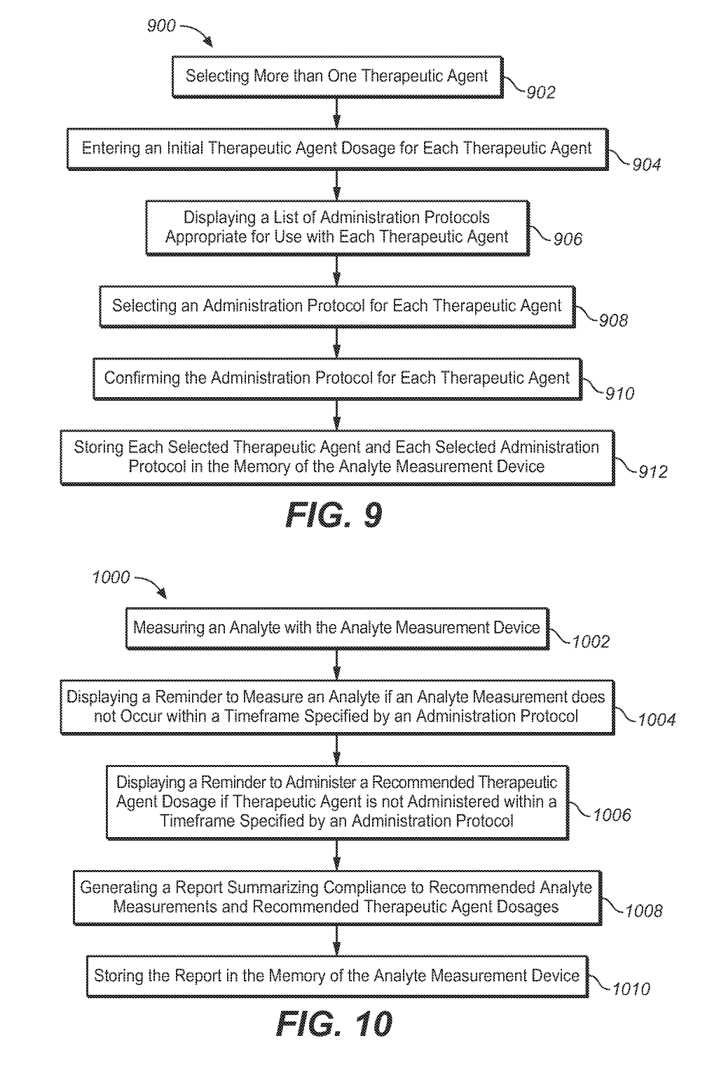

FIG. 9 is an exemplary flow chart illustrating a method of operating an analyte measurement device, according to an embodiment described and illustrated herein. Method 900 comprises steps 902, 904, 906, 908, 910, and 912. Step 902 includes selecting more than one therapeutic agent. Step 904 includes entering an initial therapeutic agent dosage for each therapeutic agent. Step 906 includes displaying a list of administration protocols appropriate for use with each therapeutic agent. Step 908 includes selecting an administration protocol for each therapeutic agent. Step 910 includes confirming the administration protocol for each therapeutic agent. Step 912 includes storing each selected therapeutic agent and each selected administration protocol in the memory of the analyte measurement device. In an embodiment of the present invention, the administration protocol includes recommended times for analyte measurement. In an embodiment of the present invention, the therapeutic agents may include oral antidiabetics, GLP-1 analogues, insulin, or metabolic agents. In an embodiment of the present invention, the method further comprises prompting the user to activate measurement and dosage reminders should measurements or dosages occur outside a specified window of time.

FIG. 10 is an exemplary flow chart illustrating a method of operating an analyte measurement device, according to an embodiment described and illustrated herein. Method 1000 comprises steps 1002, 1004, 1006, 1008, and 1010. Step 1002 includes measuring an analyte with the analyte measurement device. Step 1004 includes displaying a reminder to measure an analyte if an analyte measurement does not occur within a timeframe specified by an administration protocol. Step 1006 includes displaying a reminder to administer a recommended therapeutic agent dosage if therapeutic agent is not administered within a timeframe specified by an administration protocol. Step 1008 includes generating a report summarizing compliance to recommended analyte measurements and recommended therapeutic agent dosages. Step 1010 includes storing the report in the memory of the analyte measurement device. In an embodiment of the present invention, confirmation of a recommended therapeutic agent dosage occurs manually. In an embodiment of the present invention, confirmation of a recommended therapeutic agent dosage occurs automatically. In an embodiment of the present invention, the recommended therapeutic agent dosage is administered with a pillbox, a user-activated insulin pen, a user-activated inhaler, or user-activated pump. In an embodiment of the present invention, the pillbox, insulin pen, inhaler, or pump sends an RFID signal to the analyte measurement device confirming delivery of the recommended therapeutic agent dosage.

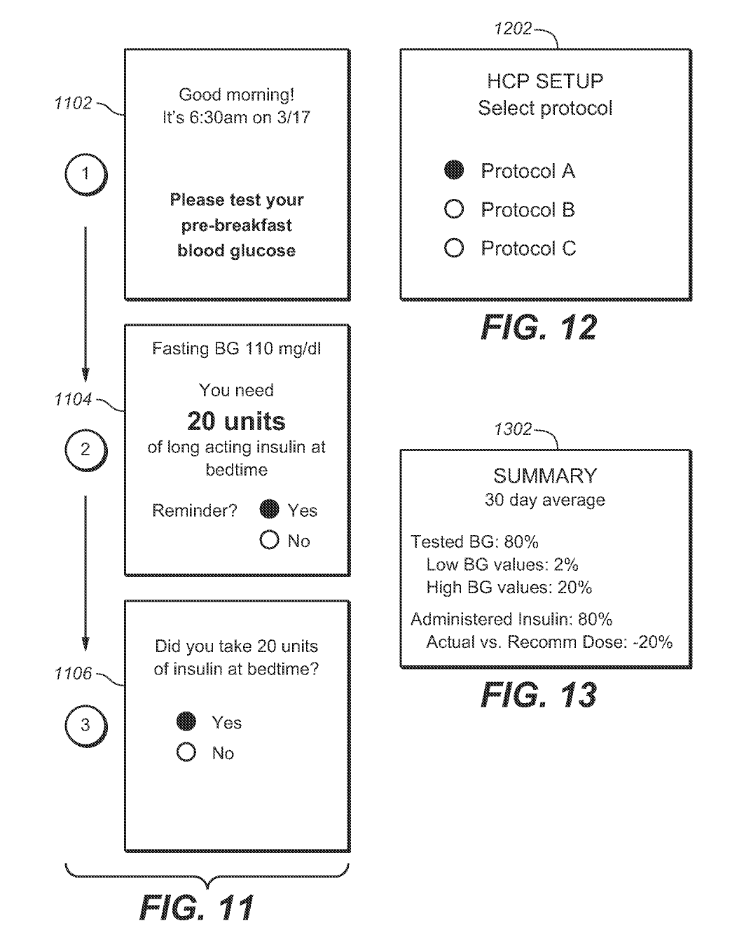

FIG. 11 illustrates a series of user interface screens displayed during a method of operating an analyte measurement device, according to an embodiment of the present invention. In screen 1102, the user is prompted to measure their pre-breakfast blood glucose. Screen 1104 displays the measured pre-breakfast (or fasting) blood glucose result, a recommended dose of insulin and its time of administration. The user is also prompted to set a reminder. Screen 1106 illustrates the reminder, displayed just before the recommended administration time.

FIG. 12 illustrates a user interface screen displayed during a method of operating an analyte measurement device, according to an embodiment of the present invention. In screen 1202, a health care practitioner or user is prompted to selects an insulin administration protocol.

FIG. 13 illustrates a user interface screen that displayed during a method of operating an analyte measurement device, according to an embodiment of the present invention. In screen 1302, a compliance summary of analyte measurement and therapeutic agent dosing over a certain time period is displayed.

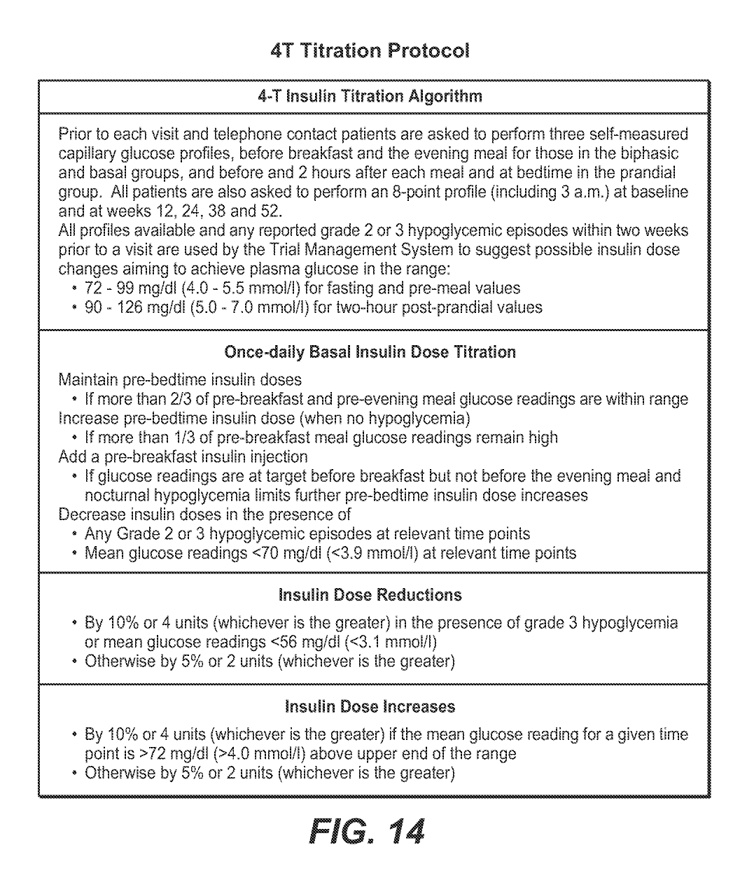

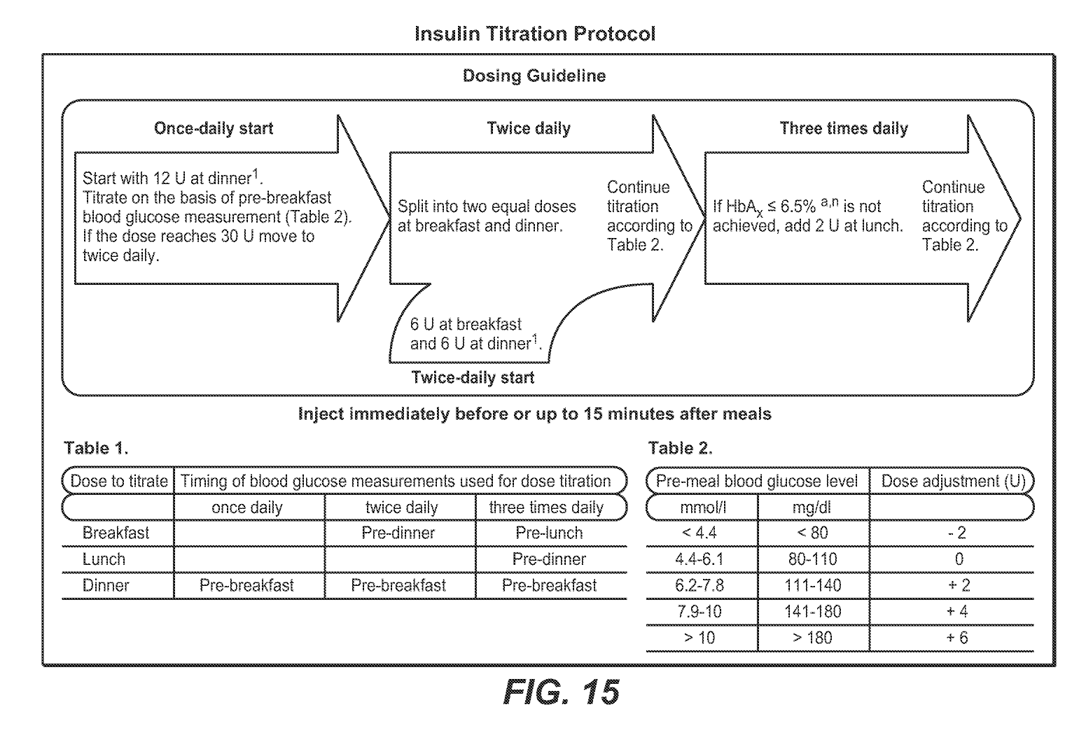

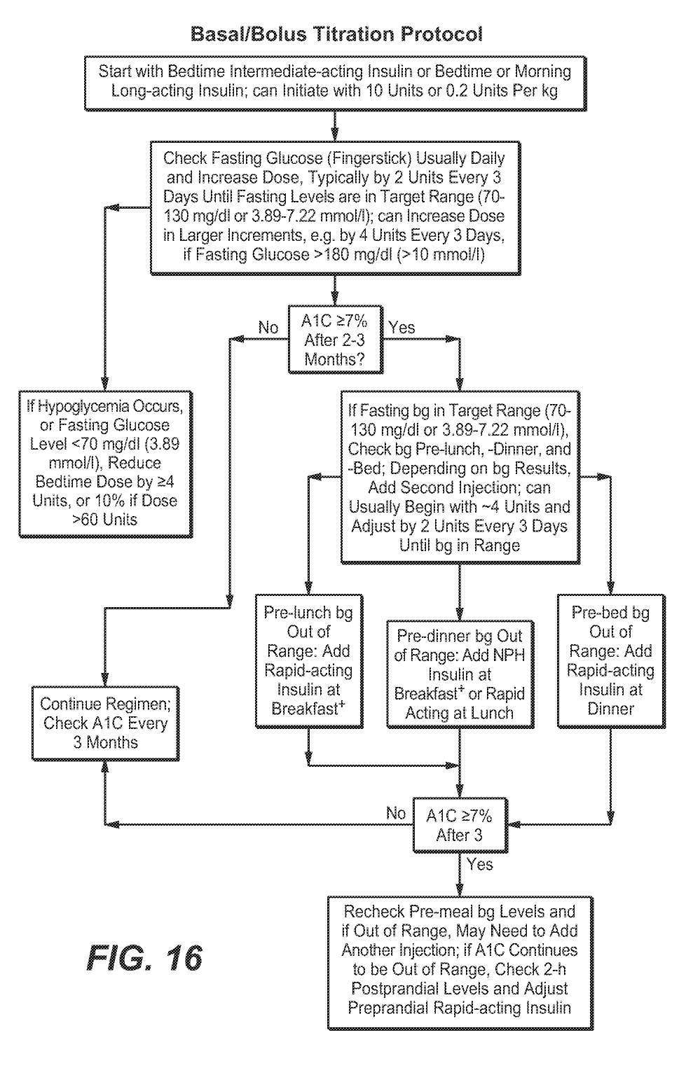

FIG. 14 illustrates an exemplary treat-to-target protocol that could be used in an embodiment of the present invention. FIG. 15 illustrates an exemplary treat-to-target protocol that could be used in an embodiment of the present invention. FIG. 16 illustrates an exemplary treat-to-target intensification protocol that could be used in an embodiment of the present invention.

FIG. 17 is a simplified block diagram of an analyte measurement and management device 1700 for use with a user-activated therapeutic agent delivery device 1799 according to an embodiment of the present invention. An analyte measurement and management device 1700 includes an analyte measurement module 1702 configured to measure an analyte (e.g., blood glucose) in a bodily fluid sample (such as blood), a memory module 1704, processor module 1706, a visual display 1708, and a delivery device communication module 1710, in addition to a user interface 1712. The analyte memory module 1702, memory module 1704, processor module 1706, visual display 1708, delivery device communication module 1710 and user interface 1712 are in operative communication with one another.

Memory module 1702 is configured for storing at least one therapeutic administration protocol while processor module 1706 is configured to calculate a recommended therapeutic agent dosage and recommended administration time for user-activated delivery of the recommended therapeutic agent dosage. Such calculations use the therapeutic administration protocol stored in memory module 1702.

In addition, visual display module 1708 is configured to display the recommended therapeutic agent dosage and recommended administration time to a user and user interface 1712 is configured for accepting user input to analyte measurement and management device 1700 via, for example, user-operated interface buttons (not shown in FIG. 17).

Delivery device communication module 1710 is configured to detect user-activated administration (i.e., delivery) of the therapeutic agent by the user-activated therapeutic agent delivery device 1799 and communicate such detection to the processor module 1706 and/or memory module 1702. Moreover, the analyte measurement module, memory module, processor module, visual display, user interface and delivery device communication module of analyte measurement and management device 1700 are integrated as a single hand-held unit such as, without limitation, the unit illustrated in FIG. 1 as element 100.

Once apprised of the present disclosure, one of skill in the art will recognize that analyte measurement and management device 1700 can be modified to perform any of the functions described above with respect to FIGS. 1 through 16 and the devices and methods associated with these Figures. Moreover, analyte measurement and management device 1700 can be configured to possess characteristics described elsewhere herein with respect to embodiments of the present invention including, for example, characteristics of the methods for operating an analyte measurement device described with respect to FIGS. 2 through 16.

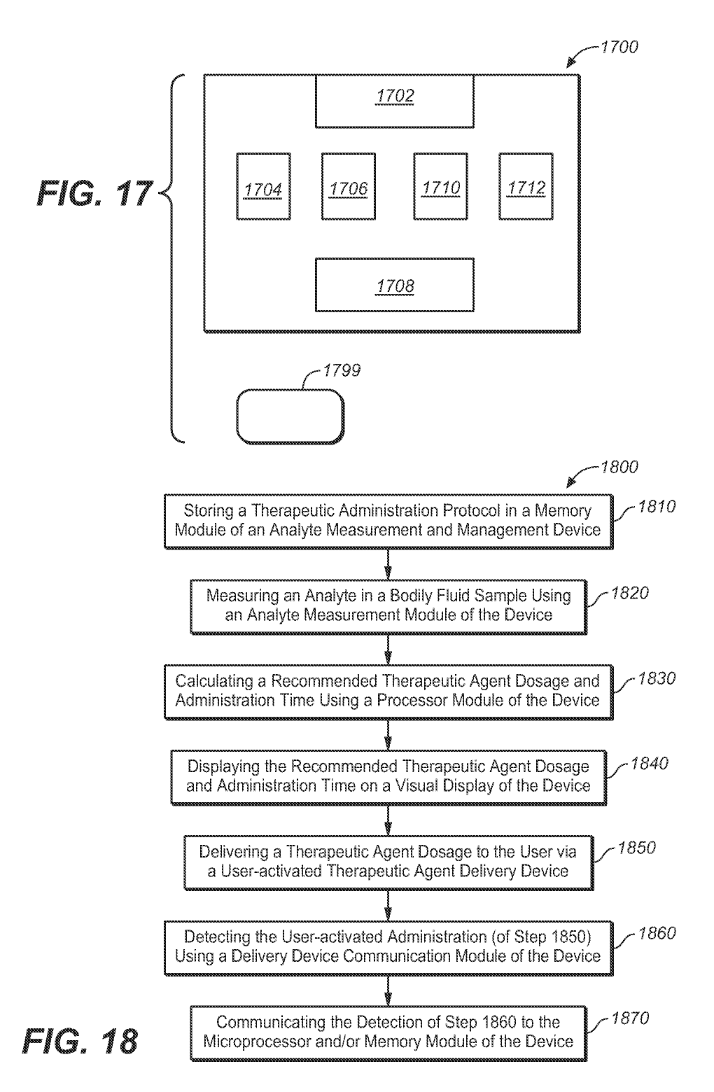

FIG. 18 is a flow diagram illustrating stages in a method 1800 for measuring and managing an analyte in a bodily fluid according to an embodiment of the present invention. Method 1800 includes, at step 1810, storing at least one therapeutic administration protocol in a memory module of an analyte measurement and management device.

Method 1800 also includes measuring the analyte in the bodily fluid sample using an analyte measurement module of the device (see step 1820 of FIG. 18) and calculating, with a processor module of the device, a recommended therapeutic agent dosage (for example, an insulin dosage) and a recommended administration time for user-activated delivery of the dosage. The calculation employs the therapeutic administration protocol stored in the memory module (as noted in step 1830 of FIG. 18).

Method 1800 further includes displaying the recommended therapeutic agent dosage and administration time to a user on a visual display of the device as noted in step 1840, delivering a therapeutic agent dosage to the user via a user-activated therapeutic agent delivery device (see step 1850), and detecting the user-activated administration (delivery) of the therapeutic agent using a delivery device communication module of the device (refer to step 1860 of FIG. 18).

Moreover, at step 1870, method 1800 further includes communicating the aforementioned detection to the processor module and/or memory module using the delivery device communication module. It should be noted that the method employs analyte measurement, memory, processor, and delivery device modules, as well as a visual display, and user interface, that are integrated as a single hand-held unit (such as the unit depicted as element 100 in FIG. 1 and analyte measurement and management device 1700 of FIG. 17).

Once apprised of the present disclosure, one of skill in the art will recognize that method 1800 can be augmented to include performance of any of the functions described above with respect to FIGS. 1 through 17 and/or to have perform steps with characteristics described elsewhere herein with respect to various embodiments of the present invention.

Embodiments of the current invention are beneficial in significantly reducing obstacles associated with initiating, maintaining and managing an analyte testing and therapeutic agent dosing regimen such as blood glucose monitoring and insulin administration. The present invention enables easy initiation and intensification, and improved compliance with a prescribed regimen by providing a simple, efficient way of guiding the patient in a step-by-step manner. By logging information on recommended versus the actual regimen followed by the patient in the manner described herein, the testing device and methods described and illustrated herein provide an effective and unitary record keeping system to help the patient and healthcare practitioner provide better care.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A method for measuring and managing an analyte in a bodily fluid, the method comprising:
storing at least one therapeutic administration protocol in a memory module of an analyte measurement and management device;
measuring an analyte in a bodily fluid sample using an analyte measurement module of the analyte measurement and management device;
calculating at least a recommended therapeutic agent dosage and a recommended administration time for user-activated delivery of the recommended therapeutic agent dosage by employing the therapeutic administration protocol stored in the memory module in a processor module of the analyte measurement and management device;
displaying at least the recommended therapeutic agent dosage and recommended administration time to a user on a visual display of the analyte measurement and management device;
delivering a therapeutic agent dosage to the user via a user-activated therapeutic agent delivery device;
detecting the user-activated administration of the therapeutic agent using a delivery device communication module of the analyte measurement and management device; and
communicating such detection to at least one of the processor module and memory module using the delivery device communication module;
wherein the analyte measurement, memory, processor, visual display, user interface and delivery device communication module are integrated as a single hand-held unit.

2. The method of claim 1 further including the step of inputting meal information by a user via a user interface of the analyte measurement and management device.

3. The method of claim 1 wherein the analyte is blood glucose and the therapeutic agent is insulin.

4. The method of claim 1 wherein the displaying step displays the recommended therapeutic agent dosage in the format of user button pushes on the user-activated therapeutic agent delivery device.

5. The method of claim 1 wherein the delivery step is accomplished via the user pushing a button on the user-activated therapeutic agent delivery device.

6. The method of claim 1 wherein the communicating step is accomplished using RFID techniques.

7. The method of claim 1 wherein the delivering step includes delivering a therapeutic agent dosage of a therapeutic agent selected from the group consisting of medications for metabolic management, hormonal therapy agents, oncology agents, pain management agents, regenerative medicine agents, and a combination thereof.

8. The method of claim 1 further including the step of confirming delivery of the therapeutic agent and actual therapeutic agent dosage by the user via a user interface of the analyte measurement and management device.

9. The method of claim 1 wherein the therapeutic administration protocol of the calculating step uses therapeutic agent type, a most recent analyte measurement value, time of the most recent analyte measurement, at least one previous analyte measurement value, at least one previous therapeutic agent dosage, and time of the at least one previous therapeutic agent dosage during the calculation.

10. The method of claim 1 wherein further including the step of generating a reporting summary by the processor module, the reporting summary including at least a percentage of detected user-activated administration in comparison to recommended therapeutic agent dosages.

11. The method of claim 10 further including the step of displaying the reporting summary on the visual display.

12. The method of claim 10 further including outputting the reporting summary from the analyte measurement and management device to an external device.

13. The method of claim 1 wherein the detecting step employs a wireless technique.

14. The method of claim 1 further including the step of generating an alarm using an alarm module of the analyte measurement and management device when the medical delivery device communication module has not detected user-activated administration of the therapeutic agent within a predetermined time window around the recommended administration time.

15. The method of claim 1 further including the step of updating the therapeutic administration protocol stored in the memory module.

16. The method of claim 1 further including the step of:
updating reporting summary software stored in the memory module.

17. The method of claim 1 further including the steps of:
retrieving a recommended therapeutic agent dosage and a recommended administration time from the memory module; and
displaying the retrieved recommended therapeutic agent dosage and the retrieved recommended administration time to user on the visual display.

18. The method of claim 1 wherein the detecting step includes detecting an actual therapeutic agent dosage and actual therapeutic agent administration time.

19. The method of claim 18 further including the step of:
storing a result of the measuring step, the recommended therapeutic agent dosage, the actual therapeutic agent dosage and the actual therapeutic agent administration time in the memory module.

20. The method of claim 1 further comprising the step of:
selecting a therapeutic administration protocol for use in the calculating step from a plurality of therapeutic administration protocols stored in the memory module.
